(19) United States
(12) Patent Application Publication
(10) Pub. No.: US 2007/0239115 A1
(43) Pub. Date: Oct. 11, 2007

(54) Co-extruded syringe assembly and method for making the same

(75) Inventor: Michael D. Cecchi, Madison, CT (US)

Correspondence Address: William W. Jones, 6 Jupiter Lane, Madison, CT, 06443, US

(73) Assignee: Genx International, Inc.

(21) Appl. No.: 11/393,415

(22) Filed: Mar. 31, 2006

(51) Int. Cl. A61M 5/00

(52) U.S. Cl. 604/187

(57) ABSTRACT

A syringe assembly is made from a co-extrusion of materials which allows a small volume of fluids or vacuum to be drawn when combined with a plunger. The co-extrusion allows the creation of a syringe assembly by extrusion allowing many varying lengths and sizes, as well as various vacuum ratios. The syringe allows deviations to the vacuum or volume of fluids with minor changes to the inner tubing member and will not change or distort the outer dimensions of the device. The syringe differs from traditional syringes in that it does not rely on exacting measurements to determine incremental movement of the plunger to determine volumes, and does not rely on the exacting conformity of such parts to determine volume. The syringe also differs from conventional syringes since conventional syringes are currently made by traditional molding techniques. The assembly will allow the syringe to be made for collecting and dispensing smaller useful volumes, and will be more ergonomically satisfactory to technicians using the syringe assembly.

### TECHNICAL FIELD

This invention relates to a syringe assembly for the creation of a vacuum or for the expelling of a volume of liquids. More particularly, this invention relates to a syringe which is formed by co-extrusion of several layers of different materials and to a method for making the same.

### BACKGROUND ART

Syringes are used for drawing or dispensing liquids, and at times are used to create a vacuum. In one application in the area of human reproduction syringes are use to create a small volume at the end of intra uterine insemination (IUI), or embryo transfer catheters. Syringes are also to draw culture media products or specimens into the syringe, or into a length of tubing, such as a catheter, which is attached to a distal end of the syringe.

The problem presented by attachment of a syringe to the end of an embryo transfer catheter is that the inner walls of the catheters may only be 0.025 mm in diameter. The individual using the catheters, in conjunction with a currently available syringe, generally uses a one cc syringe, which is typically the smallest available disposable syringe. The problem that is encountered is due to the fact that the volume of the syringes is approximately fifty times that of the embryo transfer catheters. The protocol for embryo transfer suggests that the embryos, along medium from a Petri dish be drawn into the front part of the catheter, at approximately a two centimeters long depth. The syringe will be drawn back to create an exact volume to allow the catheter to be filled two cm. This is where the problem arises. Due to the significant adverse ratio of volume between the syringe and the catheter, the technician can only draw the syringe backs one sixteenth to one eighth of an inch. The fact that the syringe may stick and not move smoothly can also interfere with the process. This makes performing this operation very difficult. It also means the technician cannot accurately or ergonomically control the amount of vacuum that must be created.

Another problem in this procedure is that it is very difficult to make a syringe that would have an equal volume and equal length of pull to the catheters. If the inner bore size of the syringe cavity would be the same as the catheter (0.025 mm) it would require a plunger of an even smaller diameter, and at this small dimension it is very difficult to manufacture the inner bore and to hold the fight tolerance and then to be able to make a workable plunger of a smaller size and also to be able to make a workable product which will maintain size tolerances and also hold a vacuum. Current syringes also are built to deliver accurate amounts of fluids and measures. The syringes are build in exacting parts that include markings on the outer walls of the device which indicate exactly how much of a vacuum or fluid is being draw into the syringe or into the attached device.

All current syringes have difficulty in drawing up the exact amount of fluids when built to be used for micro vacuums. They lack the inability to maintain dose tolerances of the materials from which they are formed due to distortions during shrinkage of the materials and an overall difficulty in manufacturing syringes of this size.

Currently syringes are made by either blow molding or injection molding the body of the syringe and by injection molding the plunger portion of the syringe by using composites or composites. The plunger contains an expanded part on an end which creates the suction when drawn. Currently there are no syringes marketed that use an extrusion technique for the making of the syringe body.

Smaller syringes are generally made of glass which are made by blowing glass materials, but are not considered disposable. The cost to make each syringe makes this prohibitive.

It would be highly desirable to provide a syringe, which can deliver a micro vacuum to be used for the aspiration of fluids into the device or into attached devices such as tubing or catheters.

### DISCLOSURE OF THE INVENTION

This invention relates to an improved making of syringes by using an extrusion method, using polymers, composites or compositions. The invention is for the extrusion of the body of the syringe and them to modify the distal end to make it compatible for attachment to other devices. The syringe of this invention may be attached to other devices such as tubing sets, catheters, or IV apparatus for fluid intervention.

The syringe is formed from an extruded polymer, composite, or composite in multiple layers, which are extruded at the same time. The outer portion of the extruded tubing will be of a rigid material, such a poly carbonate, to keep the tubing rigid or semi flexible, straight and allow it to be easily held. The middle layer will be a more elastic material similar to a silicone and the inner layer will be a lubricious material to allow the plunger assembly to easily slide within the interior. The reason for the lubricous inner material is due to the fact that the plunger assembly may be in full contact for its entire length the plunger assembly as well as a long length of the inner material. This will cause a great deal of function and may cause the plunger to bind and become locked and unable to move. The plunger assembly may be of a single diameter and need not have a bulge area or enlarged area such as conventional syringes may have at the distal end. Conventional syringes by having this expanded area are less likely to become bound and may move easier. In this invention the plunger may have this expanded area, but may not if this keeps manufacturing costs down, or if a single diameter plunger works better in certain applications. Having a more flexible or softer material in the middle section will allow the material to expand and contract with the movement of the plunger therefore to create a vacuum or suction for the drawing or expelling of fluids. The inner material may also be flexible which will allow the material to expanded and contract more easily thereby enhancing its ability to create a suction or vacuum.

Once the extruded tubing has been drawn, one end may be tipped or machined to accommodate connection to a secondary device such as tubing or a catheter. The traditional end would be a luer fitting, which has a universal dimension and taper to fit most medical devices. The end may, however, be of a specific shape or design to make it attach to a mating unique design or fitting.

The syringe of this invention will result in a device which can create a vacuum without reliance on an exacting measurement of the fluid being dispelled or drawn. The syringe will allow a vacuum to be created for a specific purpose without the manufacturing restriction of exacting measurements and relationships of the parts to create this harmony.

This invention will allow the parts to be assembled to create a vacuum for use with tubing or catheters of an inner diameter that may approximate 0.025 mm in diameter without the restriction of the current methodology of syringe type devices.

The inner plunger part of the assembly is to be used by the individual user to either push or pull the plunger to create a vacuum or dispel fluids. The plunger will ride in an inner tubular member which will allow the creation of this vacuum. This tubular member may be inserted into the body in such a way to allow the plunger to create the vacuum and as not to move during the process.

What makes this invention different from current or conventional syringes is that the interaction of the parts does not require high exacting tolerance conformity in forming the parts of the syringe, as do present syringes. The syringe of this invention is also different from conventional syringes in that the device may not require measuring increments along the outer walls of the device to be used by the individual to measure the units of fluid or vacuum that is being drawn into the device or into the attached tubing or catheter. The syringe of this invention would be usable for applications such as the transferring of embryos or sperm. The obvious advantage is that the technician may draw the thumb assembly a workable ergonomic distance, such as three centimeters, which may draw the frontal plunger in the same distance while drawing fluid into the frontal portion of the attached catheter. This syringe is different from conventional syringes in that the user will draw up into the catheter only the amount of fluid necessary or of a desired amount to capture embryos with the catheter along with culture media, for the suspension of these embryos.

Another advantage of the invention is that by changing the inner diameter of the inner tubing, the vacuum ratios will change. An example of this is as follows. For this example if the inner body has a inner diameter of 0.025 mm and the frontal portion of the plunger member, used to create the vacuum has a diameter of 0.032 mm, the vacuum created would have a volume pull related to the 0.025 mm inner diameter of the tube. The inner tubing member is of a soft, pliable material, placed in the front of the assembly. The frontal portion of the plunger member may be a rigid composite or metal of a slightly larger diameter than the inner tubal diameter to create the vacuum. By moving the plunger assembly, within the inner tube member, it will create a vacuum upon the expansion of the inner tube's diameter. The user will be observing the frontal portion of the embryo transfer catheter, while withdrawing the plunger member to create the vacuum. The user will draw into the catheter as much liquid, along with the embryos that are necessary to bring them into the frontal portion of the catheter. The user will not at this time be concerned with the length of draw by the plunger assembly. He will draw it back a sufficient distance to accomplish his task of drawing the fluids and embryo into the catheter. This illustrates the difference of the invention as it does not rely on measuring indicia along it syringe tubule member for measuring the amounts of fluid drawn. The invention also allows the user to decide on the amount of fluid drawn without the reliance on such indicia. The plunger assembly will be pushed forward when it is time to expel the fluid and embryos.

By changing the inner tubing member and changing the inner diameter of the tubing to 0.035 mm, the vacuum pull would be related to this diameter of 0.035 mm, and would pull a larger vacuum volume and more fluids than the first diameter of 0.025 mm. This would only require the plunger member to have a frontal portion of greater than 0.035 mm or approximately 0.040 mm in order to create the vacuum. This would illustrate the ability of the invention to be easily varied in the magnitude of the vacuum draw with only minor changes and would not change the outer diameter of the device.

Another advantage of this invention is that it enables the insertion of a device, such as a pipette, tubing or the like, into a frontal inner end of the interior bore of the syringe. Currently, pipette attachments utilize an adjunct collar mechanism to hold a pipette in place. This, of course, requires additional parts for the syringe.

The pipette attachment feature of this invention will be more flexible and can be used with various sizes of pipettes. The inclusion of an inner soft resilient layer in the extruded syringe body allows one to simply insert a pipette or other tube into the distal end of the syringe where the pipette or tube will be firmly gripped and sealed by the resilient layer of the syringe. The syringe likewise will be capable of such engagement with a variety of different diameter pipettes or tubes due to the elasticity of the inner layer of the syringe.

It is therefore an object of this invention to provide an improved syringe which is able to readily create a small vacuum.

It is an additional object of this invention to provide an improved syringe which is able to draw into an attached tube or catheter the amount of fluid necessary to draw up embryos or specimens in micro liquid amounts and volumes.

It is a further object of this invention to provide an improved syringe able to draw into an attached tube or catheter a desired amount of fluid while building the syringe with simpler parts and less overall dimensional tolerance.

It is another objective of this invention to overcome the traditional shortfalls of manufacturing a syringe to draw micro volumes of a vacuum or of fluids.

### BRIEF DESCRIPTION OF THE DRAWINGS

These and other objects and advantages of the invention will become more readily apparent from the following detailed description of several embodiments of the invention when taken in conjunction with the accompanying drawings in which:

FIG. 1 is a fragmented axial cross-sectional view of the body of a syringe formed in accordance with this invention;

FIG. 2 is a fragmented side elevational view of the body of the syringe of FIG. 1 showing the distal tip portion thereof;

FIG. 2A is a fragmented side elevational view of the body of the syringe similar to FIG. 2 but showing a first modification of the distal tip portion thereof;

FIG. 2B is a fragmented side elevational view of the body of the syringe similar to FIG. 2A but showing a second modification of the distal tip portion thereof;

FIG. 3 is an end elevational view of the syringe of FIG. 1;

FIG. 4 is an exploded side view of the several components of the syringe of FIG. 1;

FIG. 5 is a fragmented axially sectional view of the syringe of FIG. 1 showing the body and plunger of the syringe and showing how they interact during operation of the syringe;

FIG. 6 is a side elevational of the syringe showing the plunger prior to drawing a vacuum in the syringe;

FIG. 6A is a view similar to FIG. 6 but showing the plunger after drawing a vacuum in the syringe;

FIG. 7 is a fragmented side view of one end of the syringe of this invention having a pipette or adjunct tube pressed into the end of the syringe; and

FIG. 7A is a fragmented side sectional view of the assembly shown in FIG. 7.

### DETAILED DESCRIPTION OF SPECIFIC EMBODIMENTS OF THE INVENTION

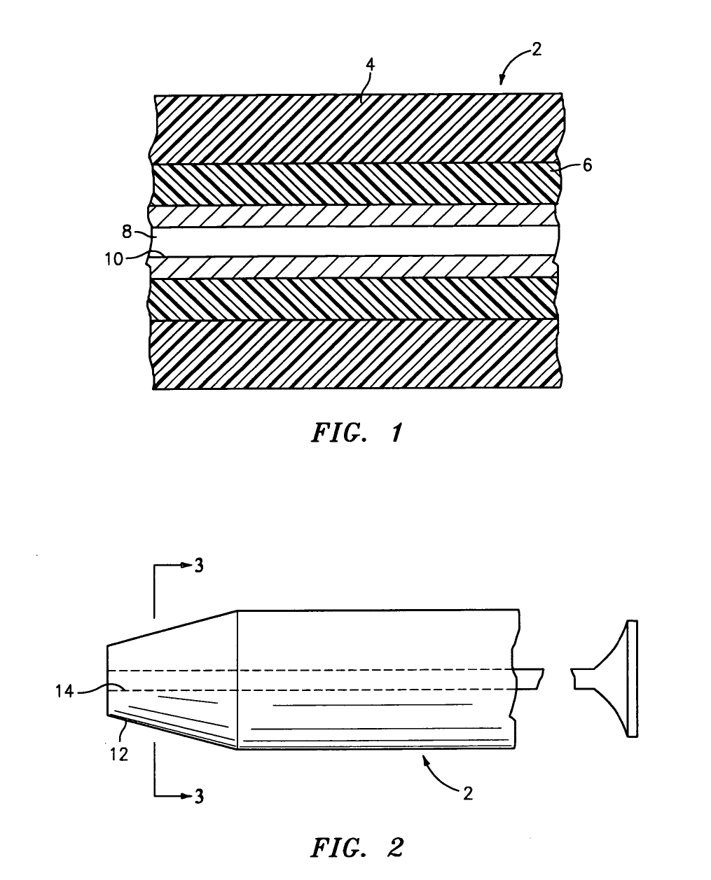

Referring now to FIG. 1, there is shown a fragmented axial cross sectional view of a preferred embodiment of a syringe body denoted generally by the numeral 2 which is formed in accordance with this invention. The syringe body 2 is preferably formed from three different materials which are co-extruded so as to form the unitary body 2 of the syringe. The outermost layer 4 is formed from a hard material such as poly carbonate. The main purpose of using this material in the outer layer 4 is to give the syringe body 2 a desirable combination of hardness and rigidity while retaining some flexibility. The medial layer 6 is formed from a softer resinous material such as urethane. The layer 6 is sufficiently flexible to be able to expand and contract when the syringe plunger (not shown) is moved back and forth through the central bore 8 of the syringe body 2. The third and innermost layer 10 of the syringe body 2 is a lubricious material such as nylon which allows the plunger to move smoothly back and forth through the bore 8 during operation of the syringe 2 and also to be sufficiently flexible to be able to expand and contract.

It will be understood that the three layers 4, 6 and 10 are simultaneously co-extruded so as to form a unified syringe body 2 which serves as the outer component of the syringe. The thicknesses of the layers 4, 6 and 10 can vary to fit the purpose of the syringe.

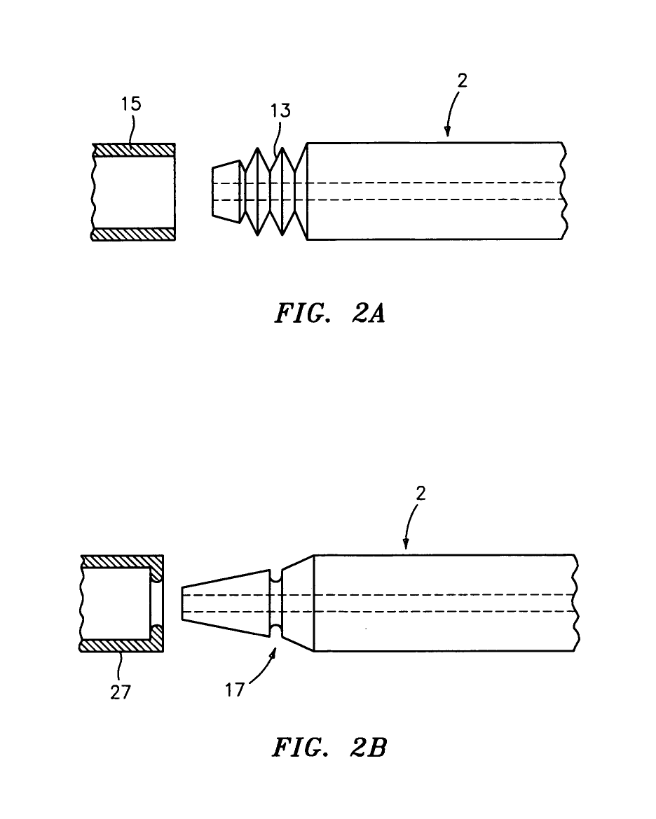

FIG. 2 is a fragmented side elevational view of the extruded syringe body 2. The syringe body 2 can be provided with a tapered tip 12 which terminates at a flat distal end surface 14. The tip 12 can be formed by machining or by heat forming. The tip 12 may be sized for a luer fitting, or can be any shape to be adapted for attachment of the syringe to an adjunct part, such as a catheter or the like.

FIGS. 2A and 2B show modifications of the tip 12 of the syringe body 2 which enhance the ability of the syringe 2 to be secured to an adjunct device. The modification shown in FIG. 2A includes serrations 13 which are machined onto the tip 12 of the syringe body 2 that will grip the interior of a tube 15 when the syringe body 2 is inserted into the tube 15. The tube 15 can be a catheter or the like which can be used in the aspiration and/or re-implantation of oocytes in an IVF procedure, or can be any other type of medical tubular device. The modification shown in FIG. 2B includes an annular recess 17 which is formed in the tip 12 of the syringe 2. The tube 15 includes an inwardly projecting annular flange 27 which will interlock with the recess 17 when the syringe body 2 is inserted into the tube 15 and will thus secure the syringe body 2 to the tube 15 during aspiration and/or re-implantation of oocytes in an IVF procedure, or other medical procedure.

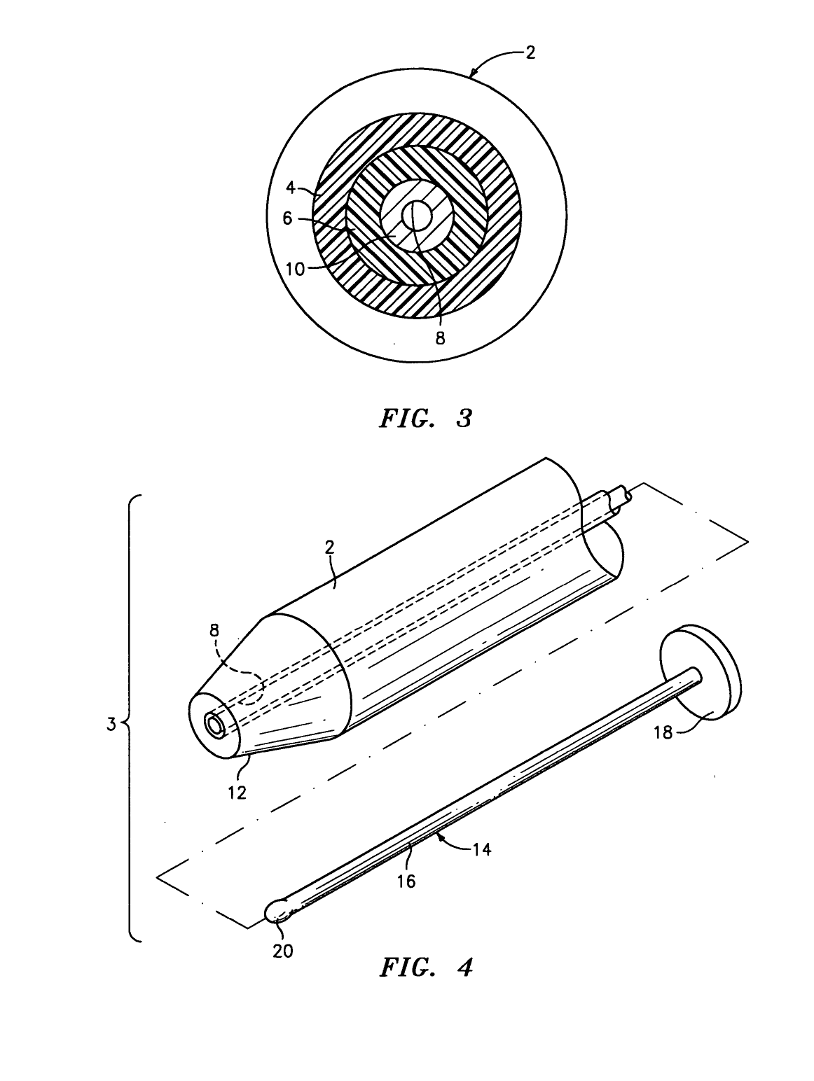

FIG. 3. Is a cross sectional view of the syringe part 2 taken along line 3-3 of FIG. 2. It will be understood that the rigid layer 4, the pliable layer 6 and the lubricious layer 10 all extend out to the distal end surface 14 of the syringe body 2. This ensures that the benefits of the layers 4, 6 and 10 are found throughout the entire length of the syringe body 2, and it also enables the tapered tip 12 of the syringe will be somewhat more flexible than the remainder of the syringe due to the thinning of the outermost rigid layer 4 in the tapered tip 12 region of the syringe.

FIG. 4 shows the two parts of a syringe assembly 3 formed in accordance with this invention. The two parts of the basic syringe 3 are the syringe body 2 and a plunger 14 which fits in the bore 8 of the syringe body 2. The plunger 14 includes a stem 16 and a thumb or finger piece 18. The distal end 20 of the stem 16 can be provided with an enlarged diameter which has a slightly larger diameter than the remainder of the stem 16. Alternatively, the stem 16 and the distal end 20 may have the same diameter.

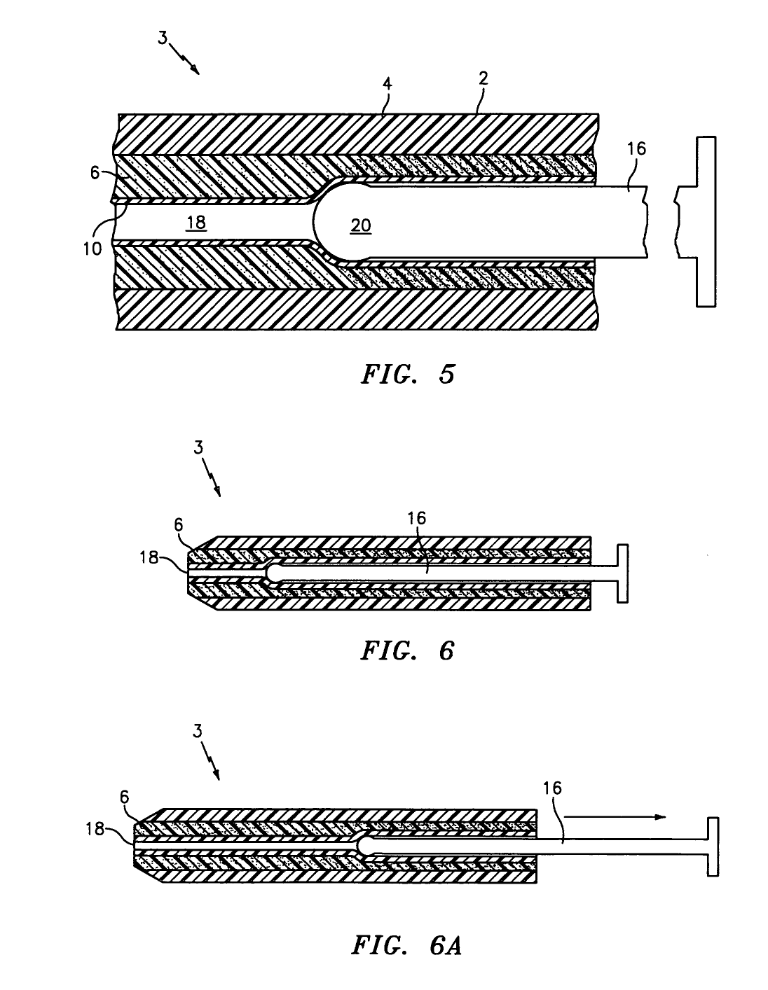

FIG. 5. illustrates the mode of operation of the syringe assembly 3. When the plunger stem 16 is inserted into the syringe bore 18, the distal end 20 of the stem 16 will compress the softer resinous layer 6 against the outer rigid layer 4 as the plunger stem 16 moves forward and backward through the bore 18 and over the lubricious layer 10. It will be appreciated that the vacuum and the counter positive pressure created by the plunger 14 as the latter moves forward and backward through the body 2 will be enhanced due to the tight fit between the distal end 20 of the stem 16 and the syringe bore 18.

FIGS. 6 and 6A illustrate two different modes of operation of the syringe assembly 3. In FIG. 6, the assembly 3 is shown with the plunger 16 in a forward or distal position in the bore 18. In FIG. 6A, the plunger 16 is shown in a more rearward position. When the plunger 16 is moved from the position shown in FIG. 6 to that shown in FIG. 6A a negative pressure or vacuum will be created in the bore 18, and when the plunger 16 is moved from the position shown in FIG. 6A to that shown in FIG. 6 a positive pressure will be created in the bore 18. These figures demonstrate how the softer layer 6 contracts and expands as the stem 16 moves through the syringe assembly 3.

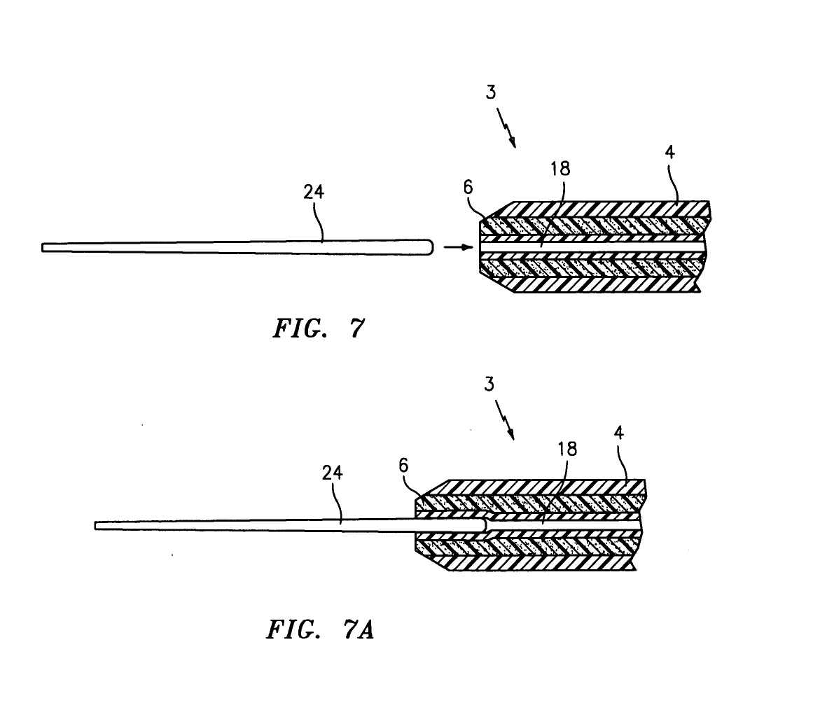

FIGS. 7 and 7A illustrate an assembly comprising the syringe 2 and a pipette or other adjunct tube 24 which is press fitted into one end of the bore 18 of the syringe 2. The tube 24 can be inserted into the syringe bore 18 and will be gripped and sealed in the bore 18 by the resilient wall component 6 of the syringe 2. The resiliency of the component 6 will create a tightly sealed joint between the syringe 2 and the tube 24 so long as the outer diameter of the tube 24 is somewhat greater than the inner diameter of the bore 18. The exact diameter of the tube 24 can vary to some extent depending on the thickness of the wall component 6 and the degree of resiliency that it possesses.

By forming the syringe body by co-extruding the several different composites the syringe bodies can be made cut from the extruded stock to any length desired. Thus the extrusion process allows the syringes to be made very small in length if so desired so that very small amounts of fluids can be drawn into or expelled from the syringes, or they can be made larger if so desired. A great deal of flexibility is achieved by forming the syringe bodies from extruded stock. For example, very small syringes can be produced which are customized so as to be able to draw or expel the small amounts of fluids which are needed for IVF clinics. These customized syringes do not require fluid volume scales printed on their exterior walls, and are ergonomically simple to use since the length of plunger movement can be made so that it equals the volume of fluid to be drawn into or expelled from the syringe. It will be readily appreciated that an improved method for making a syringe assembly and give it the ability to make a draw small volumes and to disperse small volumes. The syringe of this invention utilizes a co-extrusion method from polymers or composites, which will allow the invention to be used for micro volumes or vacuums. The syringes in this invention can be made without the high cost of molds and the molding process, can be disposable in the marketplace. Making syringes by co-extrusion will allow many various sizes to be made without the making of new molds for each size and will allow many sizes to be made at the same time.

Since many changes and variations of the disclosed embodiment of the invention may be made without departing from the inventive concept, it is not intended to limit the invention except as required by the appended claims.

What is claimed is:

1. A syringe assembly for use in drawing and/or dispensing liquids, said syringe assembly comprising:
a) an outer body part which is formed from a plurality of layers of co-extruded composites, said layers including an outer first layer formed from a first rigid composite material, an intermediate second layer formed from a composite material which is more elastic than said first composite material, and an inner third layer formed from a lubricious composite material, said third layer surrounding a through bore passage in said body part; and
b) a movable plunger part slideably disposed in said through bore passage.

2. The syringe assembly of claim 1 wherein said plunger part has a portion which is larger than said through bore passage so as to be able to temporarily compress said second layer of composite material when said plunger is moved through said through bore passage.

3. The syringe assembly of claim 1 wherein said outer first layer of said body part is formed from a poly carbonate material.

4. The syringe assembly of claim 1 wherein said intermediate second layer is formed from a soft urethane material.

5. The syringe assembly of claim 1 wherein said inner third layer is formed from a lubricious nylon material.

6. The syringe assembly of claim 2 wherein said larger portion of said plunger is at a distal end of said plunger.

7. A syringe assembly component for use in drawing and/or dispensing liquids, said component consisting of a tubular body part which is formed from a plurality of layers of co-extruded composites, said layers including an outer first layer formed from a first rigid composite material, an intermediate second layer formed from a composite material which is more elastic than said first composite material, and an inner third layer formed from a lubricious composite material, said third layer surrounding a through bore passage in said body part.

8. A method for forming a syringe assembly tubular component for use is drawing and/or dispensing liquids, said method comprising the step of co-extruding at least three separate layers of composite materials, said co-extruded separate layers including a first outer relatively rigid layer, a second intermediate relatively deformable elastic layer, and a third inner lubricious layer, said inner lubricious layer forming a lining for a through bore passage in said component.

9. A syringe assembly for use in drawing and/or dispensing liquids, said syringe assembly comprising:
a) an outer body part having a through bore passage, said outer body part being formed from a plurality of layers of co-extruded composites, said layers including an outer first layer formed from a first rigid composite material, and an inner second layer formed from a composite material which is more elastic than said first composite material; and
b) a movable plunger part slideably disposed in said through bore passage.

10. The syringe assembly of claim 9 further comprising a third inner lubricious layer, said inner lubricious layer forming a lining for said through bore passage in said component.

11. The syringe assembly of claim 9 wherein said plunger part has a portion which is larger than said through bore passage so as to be able to temporarily compress said second layer of composite material when said plunger is moved through said through bore passage.

12. The syringe assembly of claim 9 wherein said outer first layer of said body part is formed from a poly carbonate material.

13. The syringe assembly of claim 9 wherein said second layer is formed from a soft urethane material.

14. The syringe assembly of claim 10 wherein said inner third layer is formed from a lubricious nylon material.

15. The syringe assembly of claim 9 further comprising an adjunct tube, such as a pipette or the like, which is press fitted into said through bore passage and is tightly secured therein in a sealed manner by reason of the resilient elasticity of said second layer.

16. A syringe assembly for use in drawing and/or dispensing liquids, said syringe assembly comprising:
a) an outer body part having a through bore passage, said outer body part being formed from a plurality of layers of co-extruded composites, said layers including an outer first layer formed from a first rigid composite material, and an inner second layer formed from a composite material which is more elastic than said first composite material, said outer body part having a tapered end which is configured so as to provide an interlocking securement to a tubular adjunct member into which said tapered end is inserted; and
b) a movable plunger part slideably disposed in said through bore passage.

17. The syringe assembly of claim 16 wherein said tapered end of said outer body part has a serrated surface which will grip the tubular adjunct member.

18. The syringe assembly of claim 16 wherein said tapered end of said outer body part has an external annular groove which will interlock with a complementary internal annular flange on the tubular adjunct member.
